(19) United States
(12) Patent Application Publication
(10) Pub. No.: US 2005/0069504 A1
(43) Pub. Date: Mar. 31, 2005

(54) Coating for nail care having antimicrobial properties

(76) Inventor: Daniel J. Beaurline, Dallas, TX (US)

Correspondence Address: SIDLEY AUSTIN BROWN & WOOD LLP, 717 NORTH HARWOOD, SUITE 3400, DALLAS, TX, 75201, US

(21) Appl. No.: 10/496,617

(22) PCT Filed: Nov. 19, 2002
(86) PCT No.: PCT/US02/37141

Related U.S. Application Data
(60) Provisional application No. 60/334,529, filed on Nov. 30, 2001.

(51) Int. Cl. A61K 7/42; A61K 7/04

(52) U.S. Cl. 424/59; 424/61

(57) ABSTRACT

The described embodiments of the present invention include a nail coating composition substantially free to totally free of aromatic solvents, ketones, and formaldehyde-containing resins. Instead, the nail coating composition of the instant invention may include the following nitrocellulose, maleic-modified rosin based resin and polyester resin as film forming polymers; sucrose acetate isobutyrate, butyl benzyl phthalate, and glyceryl tribenzoate as plasticizers; at least one vitamin; at least one UV blocking agent; at least one protein; at least one moisturizer; at least one smoothing agent; at least one adhesion promoter; at least one antifungal/antimicrobial agent; and a mixture of solvents. The solvents in the nail coating composition are aliphatic solvents; cycloaliphatic solvents or combinations thereof. Exemplary solvents include alkanes having 4 to 10 carbon atoms, aliphatic esters having 3 to 10 carbon atoms, alkanols having 2 to 10 carbon atoms, cycloalkanes having 4 to 10 carbon atoms, cycloaliphatic esters having 4 to 10 carbon atoms, cycloalkanols having 4 to 10 carbon atoms, and mixtures thereof.

### FIELD OF THE INVENTION

The invention relates to a composition which can be applied to fingernails or toenails and provides antimicrobial properties.

### BACKGROUND OF THE INVENTION

Nail polish is generally applied to fingernails or toenails as two or more layers. The layers potentially include a base coat layer, one or more pigmented layers, and a top layer. It is desirable for each applied coat to be completely dry before the next coat is applied. To minimize the time required to complete the multi-layer nail coating, it is desirable to be able to apply a secondary coat to the base coat before the base coat is completely dry. However, if the base coat dries too slowly after the application of the secondary coat, the base coat solvents can be trapped at the interface between the secondary coat and the base coat. This reduces the cohesiveness of the base coat and the adherence of the secondary coat to the base coat. Thus, it is desirable to provide a base coat wherein each of the solvents in the base coat can evaporate from the base coat within a short period of time and thus not be trapped by a secondary coat.

It has been a common practice to employ aromatic solvents, such as toluene, as a solvent in nail coating compositions. However, the use of toluene is now considered to be undesirable because it is toxic by ingestion, inhalation, or skin absorption, and may cause mild macrocytic anemia. Many of the other aromatic solvents currently employed in nail coating compositions have a drying effect on human skin and nails during prolonged contact. Thus, it is desirable to avoid the use of aromatic solvents.

Another common element employed in nail coatings is the combination of a toluene-sulfonamide-formaldehyde resin with nitrocellulose. Nitrocellulose, or cellulose nitrate, is a thermoplastic, non-oxidizing polymer which, when cast from a solvent solution, dries by evaporation to form a film. Although commonly employed because the resulting film is relatively hard and resistant to abrasion, nitrocellulose is relatively brittle. In addition, the adhesion of the nitrocellulose coating to the nail deteriorates within a few days. Thus, nitrocellulose by itself does not provide the desired long lasting adhesion of the coating to the nail.

A combination of nitrocellulose with a toluene-sulfonamide-formaldehyde resin provides improved strength and adhesion characteristics over that offered solely by the nitrocellulose. However, the use of formaldehyde-containing resins in a nail coating is undesirable. Formaldehyde-containing resins dry the nails and make the nails brittle. In addition, allergies have also been reported for arylsulfonamide/formaldehyde resins. For example, many cases of nail enamel dermatitis are caused by toluene-sulfonamide-formaldehyde resin. Thus, there is a need for a nail coating composition that is comprised of a combination of polymers which provide the desired initial adhesion and the desired long term adhesion, while avoiding the use of formaldehyde containing polymers.

Dyer, U.S. Pat. No. 6,022,549, teaches including an antimicrobial into a nail coating. Specifically, Dyer discloses the inclusion of certain benzyl alkonium chlorides and phenols such as resorcinois and cresols in the range of 0.5 percent by weight to about 5 percent by weight. However, Dyer does not teach the inclusion of antifungal/bacteriostatic agent 2,4,4′-trichloro-2′-hydroxydiphenyl ether, commonly referred to as triclosan.

In U.S. Pat. No. 5,399,343, Smith teaches that bacteriostatic agents such as triclosan are used in the cosmetic industry, but are not suitable for use in nail polishes because triclosan has no film-forming capabilities (as noted at column 1, lines 49-65).

Sun, et al. in U.S. Pat. No. 6,231,875 disclose a topical composition for treating human nail and skin diseases including fungal infections, bacterial infections, and psoriasis. The composition may be a nail lacquer that must be comprised of at least one active agent, at least one acidifier, and at least one volatile solvent. One active agent may be triclosan, preferred acidifiers are 10% HCl or 37% HCl, and preferred volatile solvents include ethyl alcohol, isopropyl alcohol, ethyl acetate, and butyl acetate.

Accordingly, there is a need for a nail coating composition which is substantially free to totally free of aromatic solvents, ketones, and formaldehyde-containing resins. There is a need for a quickly-drying base coat composition for nails providing an antifungal/antimicrobial, a desirable level of adhesion, both initially and long term, and protection for the nail against the drying effects of solvents employed in the nail coating composition. Additionally, it is also desirable that the base coat protect the nails against damage from ingredients in the secondary coats as well as in the base coat, e.g., ingredients which cause drying of the nails and/or skin.

### SUMMARY OF THE INVENTION

The described embodiments of the present invention include a nail coating composition that is substantially free to totally free of aromatic solvents, ketones, and formaldehyde-containing resins. The nail coating composition comprises a nitrocellulose polymer, a rosin based resin, polyester, sucrose acetate isobutyrate, butyl benzyl phthalate, glyceryl tribenzoate, an antifungal/antimicrobial agent, and a mixture of aliphatic and cycloaliphatic solvents. It is presently preferred that the nail coating composition also contain at least one vitamin, at least one moisturizer, and at least one protein. The composition is particularly useful for providing a base coat.

In one embodiment of the invention, the nail coating composition comprises nitrocellulose, maleic modified rosin based resin, polyester resin, sucrose acetate isobutyrate, butyl benzyl phthalate, glyceryl tribenzoate, at least one vitamin, at least one UV blocking agent, at least one protein, at least one moisturizer, at least one smoothing agent, at least one adhesion promoter, an antifungal/antimicrobial agent and a mixture of solvents, wherein all of the solvents in the nail coating composition are selected from the group consisting of alkanes having 4 to 10 carbon atoms, aliphatic esters having 3 to 10 carbon atoms, alkanols having 2 to 10 carbon atoms, cycloalkanes having 4 to 10 carbon atoms, cycloaliphatic esters having 4 to 10 carbon atoms, and cycloalkanols having 4 to 10 carbon atoms.

In a presently preferred embodiment of the invention, the nail coating composition is free of ketones and aromatic solvents as well as formaldehyde containing resins. The nail coating composition comprises nitrocellulose resin, maleic modified rosin based resin, polyester resin, sucrose acetate isobutyrate, butyl benzyl phthalate, glyceryl tribenzoate, calcium panthothenate, panthenol, benzophenone-1, lanolin, aminomethoxysilane, hydrolyzed collagen, a polyether modified dimethylpolysiloxane copolymer, ethyl acetate, n-butyl acetate, n-butanol, isopropyl alcohol, triclosan, and a naphthenic material, wherein the naphthenic material is a mixture of paraffins and cycloparaffins containing less than 1 percent aromatics.

### DETAILED DESCRIPTION OF THE INVENTION

The terms “weight percent” or “percent by weight” herein refers to the percent of a particular component in a composition based upon the total weight of the composition. These terms may be abbreviated as “wt %” or “% wt.” The terms “volume percent” or “percent by volume” herein refer to the percent of a particular component in a composition based upon the total volume of the composition. These terms may be abbreviated as “v %” or “% v.”

The term “liquid solvent” is used herein to include (a) liquid materials which are true solvents in that they dissolve the material introduced thereto, (b) liquid wetting agents, e.g., alcohols, and (c) liquid diluents, while excluding solid materials, e.g., plasticizers and secondary film forming polymers, which might have some dissolving or plasticizing effect on the film forming polymer. The terms “liquid” and “solid” indicate the physical state at 20° C. and 760 mm Hg (one atmosphere) pressure. Wetting agents can be selected to provide a favorable interaction with the primary film forming polymer. The liquid diluents can be selected to provide the desired solubility characteristics which are consistent with dissolving the film forming polymer.

The solvents for the film forming polymers in the nail coating composition of the instant invention are a mixture of acyclic aliphatic liquid solvents and cycloaliphatic liquid solvents. The acyclic aliphatic liquid solvents may be linear or branched aliphatic compounds, and the cycloaliphatic liquid solvents may be simple cycloaliphatic compounds without any branches on the rings, or branched cycloaliphatic compounds. Exemplary branched cycloaliphatic compounds are alkylcycloaliphatic compounds, dialkylcycloaliphatic compounds, trialkylcycloaliphatic compounds, and tetraalkylcycloaliphatic compounds.

Suitable aliphatic solvents include alkanes having 4 to 10 carbon atoms per molecule, aliphatic esters having 3 to 10 carbon atoms per molecule, and alkanols having 2 to 10 carbon atoms per molecule. Exemplary aliphatic esters include n-butane, isobutane, n-pentane, isopentane, hexane, heptane, isoheptane, octane, 3,3-dimethyl hexane, 3-ethyl hexane, nonane, 2,2,3-trimethyl hexane, 2-methyl octane, 3-ethyl-2-methyl hexane, 2,3-dimethyl octane, decane, methyl propionate, methyl acetate, ethyl acetate, n-propyl acetate, n-butyl acetate, isobutyl acetate, sec-butyl acetate, tert-butyl acetate, 1,1-dimethyl butyl acetate, n-propyl formate, ethyl propionate, hexyl acetate, 3-ethyl-3-pentyl acetate, octyl acetate, 2-ethyl hexyl acetate, ethanol, n-propanol, isopropanol, n-butanol, n-pentanol, n-hexanol, n-heptanol, 3-methyl 3-hexanol, 2-ethyl 3-hexanol, n-octanol, n-decanol, and mixtures of any two or more thereof. The presently preferred aliphatic esters are the acyclic hydrocarbyl esters having 3 to 6 carbon atoms per molecule, and the presently preferred alkanols are those having 3 to 6 carbon atoms per molecule. While the alkanes having 4 to 6 carbon atoms per molecule can be utilized in the invention, the presently preferred alkanes are those having 7 to 10 carbon atoms per molecule, and the more preferred alkanes are those having 8 to 9 carbon atoms per molecule.

Suitable cycloaliphatic solvents include cycloalkanes having 4 to 10 carbon atoms per molecule, cycloaliphatic esters having 4 to 10 carbon atoms per molecule, cycloalkanols having 4 to 10 carbon atoms per molecule. Exemplary cycloaliphatic esters include cyclobutane, cyclopentane, methyl cyclobutane, cyclohexane, ethyl cyclobutane, methyl cyclopentane, ethyl cyclopentane, propyl cyclopentane, 1,1,2-trimethyl cyclopentane, 1,1-dimethyl cyclohexane, 1,2-dimethyl cyclohexane, 1,3-dimethyl cyclohexane, 1,4-dimethyl cyclohexane, ethyl cyclohexane, propyl cyclohexane, isopropyl cyclohexane, 1,1,3-trimethyl cyclohexane, 1-methyl-4-ethyl cyclohexane, n-butyl cyclohexane, isobutyl cyclohexane, cyclobutanol, cyclobutylcarbinol, cyclopentanol, naphthenes, and mixtures of any two or more thereof. The presently preferred cycloalkanols are those having 4 to 6 carbon atoms per molecule. Similarly, the presently preferred cycloaliphatic esters are those having 4 to 6 carbon atoms per molecule. However, while the cycloalkanes having 4 to 6 carbon atoms per molecule can be employed in the invention, the presently preferred cycloalkanes are the branched and unbranched cycloalkanes having 7 to 10 carbon atoms per molecule, and the more preferred cycloalkanes are those having 8 to 9 carbon atoms per molecule.

Each of the solvents can be employed in any suitable amount. In general, the total amount of solvents in the nail coating composition will be in the range of about 55 to about 85 weight percent, and preferably will be in the range of about 62 to about 82 weight percent, and more preferably in the range of about 66 to about 80 weight percent, based on the total weight of the nail coating composition. In general, the cycloaliphatic solvents will constitute from about 0.8 to about 20 weight percent, preferably from about 1.5 to about 15 weight percent, and more preferably from about 2 to about 10 weight percent, based on the total weight of the nail coating composition. A presently preferred solvent mixture (including the solvents present in the polymeric ingredients) comprises ethyl acetate in the range of about 25 to about 45 weight percent, preferably in the range of about 30 to about 40 weight percent; n-butyl acetate in the range of about 15 to about 32 weight percent, preferably in the range of about 20 to about 28 weight percent; n-butanol in the range of about 0.5 to about 10 weight percent, preferably in the range of about 1 to about 5 weight percent; isopropanol in the range of about 1 to about 12 weight percent, preferably in the range of about 3 to about 10 weight percent; and napthenic material in an amount in the range of about 1 to about 20 weight percent, and preferably in the range of about 2 to about 10 weight percent, with the napthenic material having an alkane content in the range of about 10 to about 90 volume percent and a cycloalkane content in the range of about 90 to about 10 volume percent; with each of the weight percentages being based on the total weight of the nail coating composition.

Many of the commercially available naphthenic materials are petroleum refinery product streams composed of acyclic alkanes and cycloalkanes (naphthenes) having from 6 to 10 carbon atoms per molecule. This naphthenic material is comprised of from about 10 to about 90 volume percent of acyclic alkanes constituting and about 90 to about 10 volume percent cycloalkanes, with the aromatic content being less than 1 volume percent. It is presently preferred that at least 70, more preferably at least 80, and even more preferably at least 90, volume percent of the naphthenic material be acyclic alkanes and cycloalkanes containing 8 to 9 carbon atoms per molecule. The alkane content of the naphthenic material is in the range of about 25 to about 90 volume percent and more preferably in the range of about 30 to 50 volume percent, the cycloalkane content being in the range of about 75 to about 10 volume percent and more preferably in the range of about 70 to about 50 volume percent, and the aromatic content being less than about 0.1 volume percent and more preferably less than about 0.01 volume percent of the naphthenic material. The naphthenic material can be a Naphtholite™ naphthenic material which is available from Union 76 Chemicals as a mixture of paraffins and cycloparaffins containing less than 1 percent aromatics, with the paraffin content being in the range of about 37 to about 50 percent and the cycloparaffin content being in the range of about 62 to about 49 percent, with at least about 90 volume percent of the paraffins and cycloparaffins having 8 to 9 carbon atoms per molecule. The naphthenic material can be a KERMAC™ VM&P Naphtha, Rule 66, which is available from Kerr-McGee Refining Corporation as a light aliphatic solvent naphtha, containing a mixture of acyclic paraffins and cycloparaffins and less than 1 percent aromatics. The naphthenic material can be a light aliphatic solvent naphtha available from Shell Chemical Company as Shell VM&P Naphtha HT™, which is a complex combination of aliphatic hydrocarbons and cycloaliphatic hydrocarbons containing 8 to 9 carbon atoms per molecule with a high naphthene content and less than 0.01 volume percent aromatic hydrocarbons.

The nitrocellulose used as the primary film forming polymer in the nail coating composition of the present invention is a lacquer grade nitrocellulose, preferably of the “RS” or “SS” type nitrocellulose from the Aqualon Company, a division of Hercules, Incorporated, e.g., nitrocellulose RS ½ second, nitrocellulose RS ¼ second, nitrocellulose RS ⅛ second, nitrocellulose SS ½ second, etc. The RS type nitrocellulose contains about 11.2 to about 12.8 percent nitrogen, while the SS type nitrocellulose contains about 10.7 to about 11.2 percent nitrogen. The RS type nitrocellulose is available in many grades of viscosity, ranging from 18 centipoises to 500 sec. The viscosity of the nitrocellulose can be modified as desired by utilizing two nitrocelluloses of differing viscosities and varying the ratio of the two nitrocelluloses. A presently preferred nitrocellulose is a nitrocellulose ultra sen ½ sec which comprises 70 weight percent nitrocellulose and 30 weight percent isopropanol. While any suitable amount of the nitrocellulose can be used in the nail coating composition, the amount of the nitrocellulose (excluding the wetting alcohol) will generally be in the range of about 7 to about 20 weight percent, will preferably be in the range of about 8 to about 16 weight percent, and more preferably in the range of about 10 to about 15 weight percent, based on the total weight of the nail coating composition.

The rosin based resin which is used in the nail coating composition can be any suitable rosin based resin, but is preferably a maleated rosin polymer. A suitable maleated rosin polymer may be glycerol which is available from Union Camp Corporation as UNI-REZ.RTM. 7003 maleic modified rosin polymer. While any suitable amount of the rosin based resin can be used in the nail coating composition, the amount of the maleated rosin polymer will generally be in the range of about 1 to about 8 weight percent, preferably will be in the range of about 2 to about 6 weight percent, and more preferably in the range of about 2.5 to about 4.5 weight percent, based on the total weight of the nail coating composition. The maleated rosin polymer has high alcohol tolerance, a pale color, and promotes hardness, high gloss and adhesion.

The polyester resin employed in the nail coating composition can be any suitable polyester resin, formed by reacting a polyhydric alcohol with a polybasic acid, e.g., phthalic acid. A presently preferred polyester is UNIPLEX 670-P polyester resin, which is available from Unitex Chemical Corporation as a clear solution of at least 70% solid polyester resin in butyl acetate solvent, having a Brookfield viscosity at 25° C. in the range of about 3200 to about 4500 cps, and which does not contain toluene, formaldehyde, or xylene. The polyester resin promotes gloss, adhesion, flexibility, wear-resistance, and water-resistance properties. While any suitable amount of the polyester can be used in the nail coating composition, the amount of the polyester (excluding the solvent) will generally be in the range of about 0.3 to about 5 weight percent, preferably will be in the range of about 0.5 to about 4 weight percent, and more preferably in the range of about 1 to about 2.5 weight percent, based on the total weight of the nail coating composition.

The antifungal/antimicrobial agent employed in the nail coating composition should have broad spectrum antifungal/antimicrobial properties, inhibiting the growth of gram positive and gram negative strains of bacteria as well as molds and yeasts. A preferred antifungal/antimicrobial agent is triclosan, also known as 2,4,4′-trichloro-2′-hydroxydiphenyl ether and sold in the U.S. under the trade name Irgasan DP-300 by Ciba Specialty Chemicals. While any suitable amount of antifungal/antimicrobial agent may be employed, the amount of the antifungal/antimicrobial agent will generally be in the range of from 0.5 to 5 percent by weight, preferably in the range of from 1 to 3 percent by weight of the nail coating composition. Although triclosan is the preferred antifungal/antimicrobial agent in the invention herein described, those skilled in the art will recognize that other antifungal/antimicrobial compounds could also be employed in the basecoat.

In general, the nail coating composition will contain at least three plasticizers, including sucrose diacetate hexaisobutyrate, butyl benzyl phthalate, and glyceryl tribenzoate. Sucrose diacetate hexaisobutyrate, C₄₀H₆₂O₁₉, is also known as sucrose acetate isobutyrate. Any other plasticizer suitable for use in nail coating can also be employed in the present nail coating composition. Examples of additional plasticizers include organic phthalates, organic adipates, and organic phosphates, e.g., butyl benzyl phthalate, camphor, dibutyl phthalate, tricresyl phosphate, diethyl phthalate, tributyl phosphate, dibutyl glycolate, dioctyl phthalate, butyl stearate, triphenyl phosphate, dibutyl ether phthalate, acetyl tributyl citrate, glyceryl triacetate, glyceryl tribenzoate, dicyclohexyl phthalate, ethylene glycol dibenzoate, and mixtures of any two or more thereof. Each of sucrose diacetate hexaisobutyrate, butyl benzyl phthalate, and glyceryl tribenzoate can be employed in any suitable amount, but each in general will be employed in an amount in the range of about 0.5 to about 8 weight percent, and preferably in an amount in the range of about 1 to about 6 weight percent, based on the total nail coating composition.

In a preferred embodiment, the nail coating composition will contain at least one suitable vitamin, preferably a member of the vitamin B family. A presently preferred vitamin is d-panthenol, C₉H₁₉NO₄, also known as Pro-Vitamin B5 or d(+)-pantothenyl alcohol, which is available from Roche Vitamins and Fine Chemicals as dex-Panthenol. Each vitamin can be present in the nail coating composition in any suitable amount, but each generally will be in the range of about 0.001 to about 0.02 weight percent, and preferably will be in the range of about 0.003 to about 0.01 weight percent, based on the total weight of the nail coating composition.

In a preferred embodiment, the nail coating composition will contain at least one ultraviolet (UV) blocking agent, and any suitable UV blocker can be employed. However, it is presently preferred to employ at least two UV blockers having different ranges of UV wavelength blockage so as to extend the protection against UV radiation. Any suitable amount of the UV blockers can be employed, but the amount of each UV blocker will generally be in the range of about 0.001 to about 0.2 weight percent, and preferably will be in the range of about 0.01 to about 0.05 weight percent, based on the total weight of the nail coating composition. A presently preferred UV blocker is benzophenone-1.

In general, the nail coating composition will contain at least one protein, e.g., collagen, and at least one moisturizer, e.g., lanolin. In a presently preferred base coat composition, the protein and lanolin are provided in the form of PROTO-LAN 30, a water soluble, collagen/lanolin oil base emollient containing propylene glycol, lanolin oil, and hydrolyzed collagen, available from Maybrook Inc., and recommended for use in hair and skin care products. This material moisturizes the skin, leaving a soft after-feel, and is beneficial to dry, chapped skin. It also improves dry-down properties of alcoholic systems and mitigates the harsh effect of the alcohol. Each of the protein and the moisturizer can be employed in the nail coating composition in any suitable amount, but each will generally be in the range of about 0.001 to about 0.5 weight percent, and preferably will be in the range of about 0.01 to about 0.1 weight percent, based on the total weight of the nail coating composition.

In order to provide the desired characteristics of flow and level, the nail coating composition may contain a smoothing agent. The smoothing agent reduces friction, improves the flow of the nail coating composition during application, and improves the levelness and gloss of the surface of the nail coating composition upon drying. Suitable smoothing agents include silicone polymers and copolymers, polyamides, polyacrylamides, and polycarboxylic acids, and mixtures of any two or more thereof. Any suitable amount of smoothing agent can be employed, but the amount will generally be in the range of about 0.01 to about 2 weight percent, preferably in the range of about 0.1 to about 1 weight percent, based on the total weight of the nail coating composition. The presently preferred smoothing agent is a polysiloxane copolymer.

In order to provide the desired characteristics of adhesion, the nail coating composition may contain an adhesion promoter. The adhesion promoter improves the adhesion of the nail coating to the nail or to any previously applied coats. Any suitable amount of adhesion promoter can be employed, but the amount will generally be in the range of about 0.1 to about 5 weight percent, and preferably will be in the range of about 0.5 to about 3 weight percent, based on the total weight of the nail coating composition. Examples of suitable adhesion promoters which can be employed include sucrose benzoates, sucrose acetate isobutyrates, and aminoalkoxysilanes, with aminomethoxysilane being presently preferred.

In order to facilitate the introduction of the smoothing agent and the adhesion promoter into the nail coating composition, it is desirable that these components first be dispersed in a suitable solvent, preferably an alkanol having 2 to 6 carbon atoms, and the resulting solution then be added to the solution of the film forming polymer in its mixture of solvents. The presently preferred solvent for the smoothing agent and the adhesion promoter is isopropyl alcohol.

The nail coating composition of the instant application can be applied as a base coat over natural or synthetic nails to act as a primer for pigmented or unpigmented nail polishes. The base coat dries to the touch in less than two minutes, and a secondary coat can then be applied to the base coat. In this way, the concentration of solvents at the interface of the base coat and the secondary coat is reduced sufficiently so that adequate adhesion of the secondary coat to the base coat is achieved. This base coat of the invention provides excellent flexibility, excellent initial adhesion to the bare nail as well as excellent adhesion for at least seven days, good water resistance, excellent hardness, good strengthening, good holdout (ability of coating to provide a glossy, non-porous surface), good UV protection, calcium nutrient for the nails, and good epidermal moisturizing.

In accordance with one embodiment of the present invention, the components are mixed together in the amounts shown in the table below.

| | | Percent by | | Percent by |
|---|---|---|---|---|
| | 1 Gallon | Total | 1 Gallon | weight of total |
| | Quantity | Volume | Quantity | composition |
| Ingredient | in Ounces | (v %) | in Pounds | (wt %) |
| Ethyl Acetate | 53.38 | 41.70 | 3.02 | 37.66 |
| n-Butyl Acetate | 36.22 | 28.38 | 2.02 | 25.12 |
| n-Butanol | 5.96 | 4.66 | 0.35 | 4.39 |
| Benzophenone-1 | 0.0024 | 0.002 | 0.0002 | 0.002 |
| Lowlite 24 | | | | |
| Nitrocellulose | 16.84 | 13.16 | 1.31 | 16.33 |
| Poly Resin | 8.25 | 6.44 | 0.65 | 8.17 |
| Uniplex 670-P | | | | |
| Polysiloxane | 0.33 | 0.26 | 0.02 | 0.25 |
| Co-polymer | | | | |
| BYK 301 | | | | |
| Butyl Benzyl | 6.93 | 5.41 | 0.49 | 6.12 |
| Pthalate Sant 160 | | | | |
| Irgasan DP 300 | 1.57 | 1.23 | 0.16 | 1.96 |

The composition is prepared by weighing and charging into a mixing vessel the ethyl acetate, n-butyl acetate, and n-butanol. The charged mixing vessel then undergoes low agitation. Added to the mixing vessel under low agitation is the appropriate amount of benzophenone-1 lowlite 24 and this mixture is allowed to agitate for five minutes. Irgasan is then added in the appropriate amount and the mixture is agitated for an additional five minutes. The agitation is then increased to high agitation and the nitrocellulose is added. The mixture is agitated for five minutes. Still under high agitation, the poly resin uniplex is added followed by the polysiloxane co-polymer and the butyl benzyl phthalate. This mixture is then allowed to agitate for an additional 20 minutes until all solids have dissolved.

The resulting composition can be applied to the nails of the user as any other nail polish or basecoat may be applied.

Although specific embodiments of the present invention are described herein, they are not to be construed as limiting the scope of the invention. Many variants of the invention will become apparent to those skilled in the art in light of the teaching of this written description. The scope of the invention is only limited by the claims appended hereto.

1. A nail coating composition comprising:
at least one solvent chosen from the group of aliphatic solvents, cycloaliphatic solvents and combinations thereof, nitrocellulose;
rosin based resin;
polyester resin;
at least three plasticizers; and
an antifungal/antimicrobial agent.

2. A nail coating composition as in claim 1, further comprising a vitamin.

3. A nail coating composition as in claim 1, further comprising a protein.

4. A nail coating composition as in claim 1, further comprising a moisturizing agent.

5. A nail coating composition as in claim 1, further comprising a smoothing agent.

6. A nail coating composition as in claim 1, further comprising an adhesion promoter.

7. A nail coating composition as in claim 1, further comprising at least one UV blocking agent.

8. A nail coating composition as in claim 1, wherein said solvent is an aliphatic solvent and comprises about 1 to 20 percent by weight, wherein the percentage by weight is based on the total weight of the nail coating composition.

9. A nail coating composition as in claim 1, wherein said solvent is an aliphatic solvent and is selected from the group comprising alkanes with 4 to 10 carbon atoms per molecule, aliphatic esters with 3 to 10 carbon atoms per molecule, alkanols with 2 to 10 carbon atoms per molecule, and mixtures of any two or more thereof.

10. A nail coating composition as in claim 8, wherein said aliphatic solvent is selected from the group comprising n-butane, isobutane, n-pentane, isopentane, hexane, heptane, isoheptane, octane, 3,3-dimethyl hexane, 3-ethyl hexane, nonane, 2,2,3-trimethyl hexane, 2-methyl octane, 3-ethyl-2-methyl hexane, 2,3-dimethyl octane, decane, methyl propionate, methyl acetate, ethyl acetate, n-propyl acetate, n-butyl acetate, isobutyl acetate, sec-butyl acetate, tert-butyl acetate, 1,1-dimethyl butyl acetate, n-propyl formate, ethyl propionate, hexyl acetate, 3-ethyl-3-pentyl acetate, octyl acetate, 2-ethyl hexyl acetate, ethanol, n-propanol, isopropanol, n-butanol, n-pentanol, n-hexanol, n-heptanol, 3-methyl 3-hexanol, 2-ethyl 3-hexanol, n-octanol, n-decanol, and mixtures of any two or more thereof.

11. A nail coating composition as in claim 1, wherein said solvent is a cycloaliphatic solvent and comprises about 0.8 to 20 percent by weight of the nail coating composition, wherein the percentage by weight is based on the total weight of the nail coating composition.

12. A nail coating composition as in claim 1, wherein said solvent is a cycloaliphatic solvent and is selected from the group comprising cycloalkanes with 4 to 10 carbon atoms per molecule, cycloaliphatic esters with 4 to 10 carbon atoms per molecule, cycloalkanols with 4 to 10 carbon atoms per molecule, and mixtures of any two or more thereof.

13. A nail coating composition as in claim 12, wherein said cycloaliphatic solvent is selected from the group comprising cyclobutane, cyclopentane, methyl cyclobutane, cyclohexane, ethyl cyclobutane, methyl cyclopentane, ethyl cyclopentane, propyl cyclopentane, 1,1,2-trimethyl cyclopentane, 1,1-dimethyl cyclohexane, 1,2-dimethyl cyclohexane, 1,3-dimethyl cyclohexane, 1,4-dimethyl cyclohexane, ethyl cyclohexane, propyl cyclohexane, isopropyl cyclohexane, 1,1,3-trimethyl cyclohexane, 1-methyl-4-ethyl cyclohexane, n-butyl cyclohexane, isobutyl cyclohexane, cyclobutanol, cyclobutylcarbinol, cyclopentanol, naphthenes, and mixtures of any two or more thereof.

14. A nail coating composition as in claim 1, wherein said solvent comprises about 55 to 85 percent by weight of the nail coating composition, wherein the percentage by weight is based on the total weight of the nail coating composition.

15. A nail coating composition as in claim 1, wherein said nitrocellulose comprises about 7 to 20 percent by weight of the nail coating composition, wherein weight percentage is based on the total weight of the nail coating composition.

16. A nail coating composition as in claim 15, wherein said nitrocellulose comprises about 11.2% to 12.8% nitrogen.

17. A nail coating composition as in claim 15, wherein said nitrocellulose comprises about 70 percent by weight nitrocellulose and about 30 percent by weight isopropanol.

18. A nail coating composition as in claim 1, wherein said rosin based resin comprises about 1 to 8 percent by weight of the nail coating composition, wherein weight percentage is based on the total weight of the nail coating composition.

19. A nail coating composition as in claim 18, wherein said rosin based resin is a maleated rosin polymer.

20. A nail coating composition as in claim 1, wherein said polyester resin comprises about 0.3 to 5 percent by weight of the nail coating composition, wherein weight percentage is based on the total weight of the nail coating composition.

21. A nail coating composition as in claim 1, wherein said plasticizers comprise a total of about 0.5 to 8 percent by weight of the nail coating composition, wherein weight percentage is based on the total weight of the nail coating composition.

22. A nail coating composition as in claim 21, wherein said plasticizers are selected from the group comprising sucrose diacetate hexaisobutyrate, butyl benzyl phthalate, glyceryl tribenzoate, butyl benzyl phthalate, camphor, dibutyl phthalate, tricresyl phosphate, diethyl phthalate, tributyl phosphate, dibutyl glycolate, dioctyl phthalate, butyl stearate, triphenyl phosphate, dibutyl ether phthalate, acetyl tributyl citrate, glyceryl triacetate, glyceryl tribenzoate, dicyclohexyl phthalate, ethylene glycol dibenzoate, and mixtures of any two or more thereof.

23. A nail coating composition as in claim 1, wherein said antimicrobial/antifungal agent comprises about 0.5 to 5 percent by weight of the nail coating composition, wherein weight percentage is based on the total weight of the nail coating composition.

24. A nail coating composition as in claim 23, wherein said antimicrobial/antifungal agent is 2,4,4′-trichloro-2′-hydroxydiphenyl ether.

25. A nail coating composition as in claim 2, wherein said vitamin comprises about 0.001 to 0.02 percent by weight of the nail coating composition, wherein weight percentages are based on the total weight of the nail coating composition.

26. A nail coating composition as in claim 25, wherein said vitamin is preferably a member of the vitamin B family.

27. A nail coating composition as in claim 3, wherein said protein comprises about 0.001 to 0.05 percent by weight of the nail coating composition, wherein weight percentages are based on the total weight of the nail coating composition.

28. A nail coating composition as in claim 27, wherein said protein is preferably collagen.

29. A nail coating composition as in claim 4, wherein said moisturizer comprises about 0.001 to 0.5 percent by weight of the nail coating composition, wherein weight percentage is based on the total weight of the nail coating composition.

30. A nail coating composition as in claim 29, wherein said moisturizer is lanolin.

31. A nail coating composition as in claim 5, wherein said smoothing agent comprises about 0.01 to 2 percent by weight of the nail coating composition, wherein weight percentages are based on the total weight of the nail coating composition.

32. A nail coating composition as in claim 31, wherein said smoothing agent is chosen from the group comprising silicone polymers and copolymers, polyamides, polyacrylamides, and polycarboxylic acids, and mixtures of any two or more thereof.

33. A nail coating composition as in claim 6, wherein said adhesion promoter comprises about 0.1 to 5 percent by weight of the nail coating composition, wherein weight percentage is based on the total weight of the nail coating composition.

34. A nail coating composition as in claim 33, wherein said adhesion promoter is selected from the group comprising sucrose benzoates, sucrose acetate isobutyrates, and aminoalkoxysilanes.

35. A nail coating composition as in claim 7, wherein each said UV blocking agent comprises about 0.001 to 0.2 percent by weight of the nail coating composition, wherein weight percentage is based on the total weight of the nail coating composition.

36. A nail coating composition as in claim 27, wherein a preferred UV blocking agent is benzophenone-1.

37. A nail coating composition as in claim 1 wherein said antifungal/antimicrobial agent is triclosan.

38. A nail coating composition comprising:
at least one aliphatic solvent;
nitrocellulose;
rosin based resin;
polyester resin;
at least three plasticizers; and
an antifungal/antimicrobial agent.

39. A nail coating composition as in claim 38, wherein said aliphatic solvent is selected from the group comprising n-butane, isobutane, n-pentane, isopentane, hexane, heptane, isoheptane, octane, 3,3-dimethyl hexane, 3-ethyl hexane, nonane, 2,2,3-trimethyl hexane, 2-methyl octane, 3-ethyl-2-methyl hexane, 2,3-dimethyl octane, decane, methyl propionate, methyl acetate, ethyl acetate, n-propyl acetate, n-butyl acetate, isobutyl acetate, sec-butyl acetate, tert-butyl acetate, 1, 1-dimethyl butyl acetate, n-propyl formate, ethyl propionate, hexyl acetate, 3-ethyl-3-pentyl acetate, octyl acetate, 2-ethyl hexyl acetate, ethanol, n-propanol, isopropanol, n-butanol, n-pentanol, n-hexanol, n-heptanol, 3-methyl 3-hexanol, 2-ethyl 3-hexanol, n-octanol, n-decanol, and mixtures of any two or more thereof.

40. A nail coating composition as in claim 38 wherein said antifungal/antimicrobial agent is triclosan.

41. A nail coating composition comprising:
at least one cycloaliphatic solvent;
nitrocellulose;
rosin based resin;
polyester resin, at least three plasticizers; and
an antifungal/antimicrobial agent.

42. A nail coating composition as in claim 41, wherein said cycloaliphatic solvent is selected from the group comprising cyclobutane, cyclopentane, methyl cyclobutane, cyclohexane, ethyl cyclobutane, methyl cyclopentane, ethyl cyclopentane, propyl cyclopentane, 1,1,2-trimethyl cyclopentane, 1,1-dimethyl cyclohexane, 1,2-dimethyl cyclohexane, 1,3-dimethyl cyclohexane, 1,4-dimethyl cyclohexane, ethyl cyclohexane, propyl cyclohexane, isopropyl cyclohexane, 1,1,3-trimethyl cyclohexane, 1-methyl-4-ethyl cyclohexane, n-butyl cyclohexane, isobutyl cyclohexane, cyclobutanol, cyclobutylcarbinol, cyclopentanol, naphthenes, and mixtures of any two or more thereof.

43. A nail coating composition as in claim 41 wherein said antifungal/antimicrobial agent is triclosan.

44. A nail coating composition comprising:
at least one aliphatic solvent;
at least one cycloaliphatic solvent;
nitrocellulose;
rosin based resin;
polyester resin;
at least three plasticizers; and
an antifungal/antimicrobial agent.

45. A nail coating composition as in claim 44, wherein said aliphatic solvent is selected from the group comprising n-butane, isobutane, n-pentane, isopentane, hexane, heptane, isoheptane, octane, 3,3-dimethyl hexane, 3-ethyl hexane, nonane, 2,2,3-trimethyl hexane, 2-methyl octane, 3-ethyl-2-methyl hexane, 2,3-dimethyl octane, decane, methyl propionate, methyl acetate, ethyl acetate, n-propyl acetate, n-butyl acetate, isobutyl acetate, sec-butyl acetate, tert-butyl acetate, 1, 1-dimethyl butyl acetate, n-propyl formate, ethyl propionate, hexyl acetate, 3-ethyl-3-pentyl acetate, octyl acetate, 2-ethyl hexyl acetate, ethanol, n-propanol, isopropanol, n-butanol, n-pentanol, n-hexanol, n-heptanol, 3-methyl 3-hexanol, 2-ethyl 3-hexanol, n-octanol, n-decanol, and mixtures of any two or more thereof.

46. A nail coating composition as in claim 44, wherein said cycloaliphatic solvent is selected from the group comprising cyclobutane, cyclopentane, methyl cyclobutane, cyclohexane, ethyl cyclobutane, methyl cyclopentane, ethyl cyclopentane, propyl cyclopentane, 1,1,2-trimethyl cyclopentane, 1,1-dimethyl cyclohexane, 1,2-dimethyl cyclohexane, 1,3-dimethyl cyclohexane, 1,4-dimethyl cyclohexane, ethyl cyclohexane, propyl cyclohexane, isopropyl cyclohexane, 1,1,3-trimethyl cyclohexane, 1-methyl-4-ethyl cyclohexane, n-butyl cyclohexane, isobutyl cyclohexane, cyclobutanol, cyclobutylcarbinol, cyclopentanol, naphthenes, and mixtures of any two or more thereof.

47. A nail coating composition as in claim 44 wherein said antifungal/antimicrobial agent is triclosan.
